(19) United States
(12) Patent Application Publication
(10) Pub. No.: US 2021/0082054 A1
(43) Pub. Date: Mar. 18, 2021

(54) AUTOMATED INSURANCE CLAIM EVALUATION THROUGH CORRELATED METADATA

(71) Applicant: International Business Machines Corporation, Armonk, NY (US)

(72) Inventors: Cesar Augusto Rodriguez Bravo, Alajuela (CR); Ivonne Rocio Cuervo Fajardo, San Rafael (CR); Ugo Ivan Orellana, Westlake, CA (US); Craig M. Trim, Ventura, CA (US)

(21) Appl. No.: 16/571,624

(22) Filed: Sep. 16, 2019

(51) Int. Cl. G06Q 40/08; G16H 40/67

(52) U.S. Cl. CPC G06Q 40/08; G16H 40/67

(57) ABSTRACT

Technology for leveraging machine learning to streamline and automate insurance claim evaluations by connecting various data sources relevant to an insurance claim, including metadata from various smart devices, to identify reliable information corroborated by multiple sources and generate objective scoring values associated with parties submitting insurance claims. Output from the leveraged machine learning techniques can be used to automatically output an insurance claim determination or provide enhanced information to an insurance providing entity through a graphical user interface (GUI) to augment and assist in making such a determination.

### BACKGROUND

The present invention relates generally to the field of fraud detection, and more particularly to fraudulent insurance claim detection.

Insurance is a means of protection from financial loss. It is a form of risk management, primarily used to hedge against the risk of a contingent or uncertain loss. An insurance providing entity is often known as an insurer or insurance company. A person or entity that purchases insurance is known as an insured or, alternatively, as a policyholder. The transaction involves the insured providing payment to the insurer in exchange for the insurer's promise to compensate the insured in the event of a covered loss. The loss typically involves something in which the insured has an insurable interest established by ownership, possession, and/or a pre-existing relationship. The insured receives a contract, known as an insurance policy, which details the conditions and circumstances under which the insurer will compensate the insured. The amount of money charged by the insurer for the coverage established in the insurance policy is called the premium. If the insured experiences a loss which is potentially covered by the insurance policy, the insured submits a claim to the insurance company for processing.

Insurance fraud is an act committed to defraud one or more insurance processes. Insurance fraud may occur when a claimant attempts to fraudulently obtain some benefit or advantage they are not legally entitled to obtain. Insurance fraud may also occur when an insurer knowingly denies one or more benefits that the insurer is contractually obligated to provide to a claimant. Common insurance fraud schemes include premium diversion, fee churning, asset diversion, and/or workers compensation fraud. False insurance claims are insurance claims filed with fraudulent intention towards an insurance provider. Fraudulent claims account for a significant portion of all claims received by insurers and cost upwards of billions of dollars annually. Insurance fraud is diverse crime that occurs across a wide range of insurance types and vary in severity. Insurance fraud poses a significant problem for the general public, governments and other organizations attempt to deter such activity when possible.

A “smart device” is an electronic device that is typically connected to other devices and/or networks through various wireless protocols (e.g., Bluetooth, Wi-Fi, etc.) that operates, to some extent, interactively and autonomously. Examples of smart devices include smartphones, autonomous vehicles, smartwatches, and smart speakers. A smart device may be programmed to complete a specific task or interact with other smart device accessories to complete tasks. Typically, data is transmitted and/or received though various wireless protocols with a wide range of applications, such as data analytics.

### SUMMARY

According to an aspect of the present invention, there is a method, computer program product and/or system that performs the following operations (not necessarily in the following order): (i) receiving an insurance event data set, including a plurality of event metadata values; (ii) parsing the event metadata values into a plurality of event data categories; (iii) generate an initial network of correlations between at least some event metadata values within the same event data category; and (iv) generate a secondary network of correlations between at least some event metadata values, where connections are made between event metadata values of different event data categories based, at least in part, on a nature of information corresponding to the event metadata values.

### BRIEF DESCRIPTION OF THE DRAWINGS

FIG. 1 is a block diagram view of a first embodiment of a system according to the present invention;

FIG. 2 is a flowchart showing a first embodiment method performed, at least in part, by the first embodiment system;

FIG. 3 is a block diagram showing a machine logic (for example, software) portion of the first embodiment system;

FIG. 4A is a block diagram showing information that is helpful in understanding the first embodiment of the present invention;

FIG. 4B is a block diagram showing information that is helpful in understanding the first embodiment of the present invention;

FIG. 5 is a screenshot view generated by the first embodiment system;

FIG. 6A is a block diagram helpful in understanding a second embodiment of the present invention;

FIG. 6B is a block diagram helpful in understanding the second embodiment of the present invention; and

FIG. 7 is a table showing information that is helpful in understanding embodiments of the present invention.

### DETAILED DESCRIPTION

Embodiments of the present invention leverage machine learning techniques to streamline and automate insurance claim evaluations by connecting various data sources relevant to an insurance claim, including metadata from various smart devices, to identify reliable information corroborated by multiple sources and generate objective scoring values associated with parties submitting insurance claims. Output from the leveraged machine learning techniques can be used to automatically output an insurance claim determination or provide enhanced information to an insurance providing entity through a graphical user interface (GUI) to augment and assist in making such a determination. This Detailed Description section is divided into the following sub-sections: (i) The Hardware and Software Environment; (ii) Example Embodiment; (iii) Further Comments and/or Embodiments; and (iv) Definitions.

### I. The Hardware and Software Environment

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

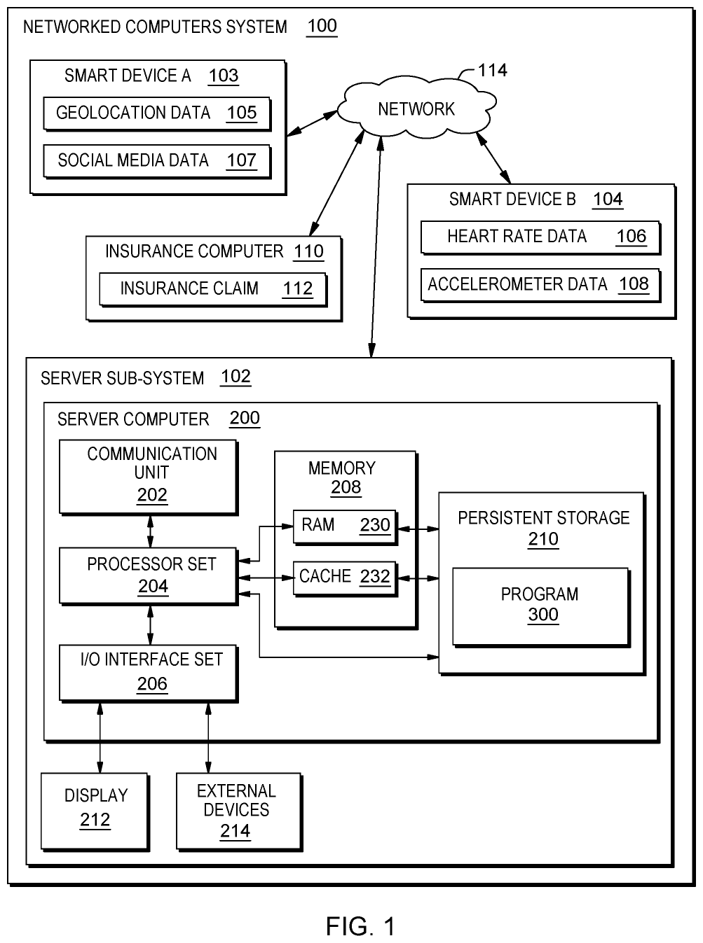

An embodiment of a possible hardware and software environment for software and/or methods according to the present invention will now be described in detail with reference to the Figures. FIG. 1 is a functional block diagram illustrating various portions of networked computers system 100, including: server sub-system 102; smart device A 103; smart device B 104; geolocation data 105; heart rate data 106; social media data 107; accelerometer data 108; insurance computer 110; insurance claim 112; communication network 114; server computer 200; communication unit 202; processor set 204; input/output (I/O) interface set 206; memory device 208; persistent storage device 210; display [device] 212; external devices [set] 214; random access memory (RAM) devices 230; cache memory device 232; and program 300.

Sub-system 102 is, in many respects, representative of the various computer sub-system(s) in the present invention. Accordingly, several portions of sub-system 102 will now be discussed in the following paragraphs.

Sub-system 102 may be a laptop computer, tablet computer, netbook computer, personal computer (PC), a desktop computer, a personal digital assistant (PDA), a smart phone, or any programmable electronic device capable of communicating with the client sub-systems via network 114. Program 300 is a collection of machine readable instructions and/or data that is used to create, manage and control certain software functions that will be discussed in detail, below, in the Example Embodiment sub-section of this Detailed Description section.

Sub-system 102 is capable of communicating with other computer sub-systems via network 114. Network 114 can be, for example, a local area network (LAN), a wide area network (WAN) such as the Internet, or a combination of the two, and can include wired, wireless, or fiber optic connections. In general, network 114 can be any combination of connections and protocols that will support communications between server and client sub-systems.

Sub-system 102 is shown as a block diagram with many double arrows. These double arrows (no separate reference numerals) represent a communications fabric, which provides communications between various components of sub-system 102. This communications fabric can be implemented with any architecture designed for passing data and/or control information between processors (such as microprocessors, communications and network processors, etc.), system memory, peripheral devices, and any other hardware components within a system. For example, the communications fabric can be implemented, at least in part, with one or more buses.

Memory 208 and persistent storage 210 are computer-readable storage media. In general, memory 208 can include any suitable volatile or non-volatile computer-readable storage media. It is further noted that, now and/or in the near future: (i) external device(s) 214 may be able to supply, some or all, memory for sub-system 102; and/or (ii) devices external to sub-system 102 may be able to provide memory for sub-system 102.

Program 300 is stored in persistent storage 210 for access and/or execution by one or more of the respective computer processors (processor set) 204, usually through one or more memories of memory 208. Persistent storage 210: (i) is at least more persistent than a signal in transit; (ii) stores the program (including its soft logic and/or data), on a tangible medium (such as magnetic or optical domains); and (iii) is substantially less persistent than permanent storage. Alternatively, data storage may be more persistent and/or permanent than the type of storage provided by persistent storage 210.

Program 300 may include both machine readable and performable instructions and/or substantive data (that is, the type of data stored in a database). In this particular embodiment, persistent storage 210 includes a magnetic hard disk drive. To name some possible variations, persistent storage 210 may include a solid state hard drive, a semiconductor storage device, read-only memory (ROM), erasable programmable read-only memory (EPROM), flash memory, or any other computer-readable storage media that is capable of storing program instructions or digital information.

The media used by persistent storage 210 may also be removable. For example, a removable hard drive may be used for persistent storage 210. Other examples include optical and magnetic disks, thumb drives, and smart cards that are inserted into a drive for transfer onto another computer-readable storage medium that is also part of persistent storage 210.

Communications unit 202, in these examples, provides for communications with other data processing systems or devices external to sub-system 102. In these examples, communications unit 202 includes one or more network interface cards. Communications unit 202 may provide communications through the use of either or both physical and wireless communications links. Any software modules discussed herein may be downloaded to a persistent storage device (such as persistent storage device 210) through a communications unit (such as communications unit 202).

I/O interface set 206 allows for input and output of data with other devices that may be connected locally in data communication with server computer 200. For example, I/O interface set 206 provides a connection to external device set 214. External device set 214 will typically include devices such as a keyboard, keypad, a touch screen, and/or some other suitable input device. External device set 214 can also include portable computer-readable storage media such as, for example, thumb drives, portable optical or magnetic disks, and memory cards. Software and data used to practice embodiments of the present invention, for example, program 300, can be stored on such portable computer-readable storage media. In these embodiments the relevant software may (or may not) be loaded, in whole or in part, onto persistent storage device 210 via I/O interface set 206. I/O interface set 206 also connects in data communication with display device 212.

Display device 212 provides a mechanism to display data to a user and may be, for example, a computer monitor or a smart phone display screen.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### II. Example Embodiment

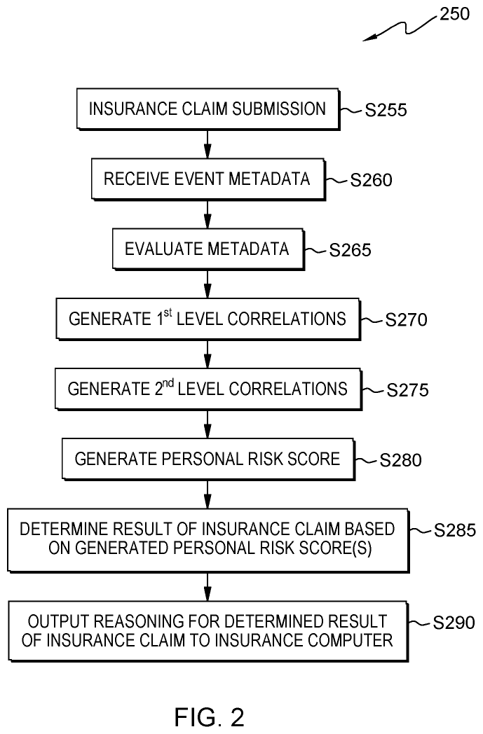

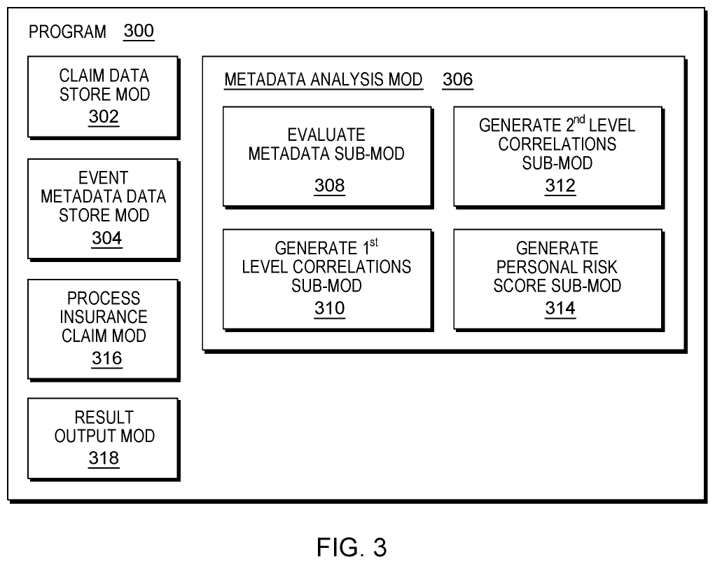

FIG. 2 shows flowchart 250 depicting a method according to the present invention. FIG. 3 shows program 300 for performing at least some of the method operations of flowchart 250. This method and associated software will now be discussed, over the course of the following paragraphs, with extensive reference to FIG. 2 (for the method operation blocks) and FIG. 3 (for the software blocks).

Processing begins at operation S255, where program 300 receives insurance claim submission data, also called “insurance claim data,” from insurance claim 112 of FIG. 1 through insurance computer 110 over network 114 and stores the insurance claim data in claim data store module (“mod”) 302. In this simplified embodiment, the insurance claim is an automobile insurance claim submitted to one insurance companies by two parties that were both involved in one accident. The two parties involved in the accident (hereinafter sometimes referred to as “party A and party B”) were involved in a bumper-to-bumper collision. That is, party A was driving behind party B, and the front bumper of party A's car hit the back bumper of party B's car (sometimes hereinafter referred to as the “insurance event”). According to the insurance claims submitted by both parties, both parties were going the speed limit on a highway, 65 miles per hour, when party B abruptly applied the car brakes which caused party A to collide with party B's car. After the accident, both parties submitted an auto insurance claim to their respective insurance companies. The insurance claim for party A indicates the following information: (i) time of event is 3:00 PM EST; (ii) date of event is Sep. 4, 2019; (iii) location of the event is between the 10^{th} and 11^{th} mile marker of route 86 in New York; (iv) other involved party is party B; (v) cause of event is party B abruptly and aggressively applied the brakes of their vehicle, suddenly slowing their velocity unexpectedly; and (vi) damage to the vehicle of party A is $5,000. The insurance claim for party B indicates the following information: (i) time of event is 3:05 PM EST; (ii) date of event is Sep. 4, 2019; (iii) location of the event is between the 10^{th} and 11^{th} mile marker of route 86 in New York; (iv) other involved party is party A; (v) cause of event is party A collided with the rear bumper of party B's car while party B braked and swerved to avoid debris on the road; and (vi) damage to the vehicle of party B is $15,000. In this simplified embodiment, both parties have individual car insurance policies with the same insurance company. The insurance company identified that the auto insurance claims from party A and party B were arose from the same car accident. The insurance company combined each insurance claim from party A and party B to form insurance claim 112. The insurance claim 112 is sent through insurance computer 110, by one or more representatives of the insurance company, to a cognitive system to identify, if any, fraudulent activity.

Alternatively, there may be two or more insurance claims sent through insurance computer 110. For example, an insurance company may have two separate claims that are transmitted to the cognitive system by insurance computer 110. As a further alternative embodiment, there may be two or more insurance companies that transmit insurance claims to the cognitive system. For example, if a car insurance accident involves two individual policy holders that have a car insurance policy with two separate insurance companies, then each insurance company may transmit the insurance claim information to the cognitive system. As a further alternative embodiment, insurance claim 112 may represent a claim for different types of insurance, such as: (a) car insurance, (b) home insurance, (c) rental insurance, (d) mortgage insurance, (e) life insurance, and (f) health insurance. For example, an insurance claim transmitted to the cognitive system may involve a life insurance policy. As a further alternative embodiment, an insurance claim transmitted to the cognitive system may involve at least one insured party. For example, an insurance company may transmit an insurance claim to the cognitive system that only involves a life insurance policy for one individual. As a yet further alternative embodiment, the insurance claims from party A and party B may be automatically linked together by a cognitive system upon parsing information from each insurance claim and identifying similar reported facts such as time, location, other party identifying information, etc.

Processing proceeds to operation S260 of FIG. 2, where event metadata data store mod 304 receives and stores metadata transmitted from smart device A 103 of FIG. 1 and metadata transmitted from smart device B 104. In this simplified embodiment, smart device A 103 is a smartphone and smart device B 104 is a smart watch. The smartphone metadata transmitted by smart device A 103 includes geolocation metadata from geolocation data 105 and social media metadata from social media data 107. The geolocation data 105 includes the GPS coordinates for the smartphone with a correlated timestamp. The social media data 107 includes any social media activity that occurred on the smartphone with a correlated timestamp. The smartwatch metadata transmitted by smart device B 104 includes heart rate metadata from heart rate data 106 and accelerometer metadata from accelerometer data 108. The heart rate data 106 includes a person's heartbeats per unit of time who was wearing the smartwatch with a correlated timestamp. The accelerometer data 108 includes changes in the angle and velocity of the smartwatch with correlated timestamps, where a change in velocity over time is considered acceleration. The smart device A 103 is a smartphone associated with party A in the car accident. In this simplified embodiment, an application is installed on smart device A 103 associated with the insurance policy of party A. Alternatively, a phone number associated with smart device A 103 may be associated with the insurance policy of party A. The smart device B 104 is a smartwatch associated with party B in the car accident. In this simplified embodiment, smart device B 104 is paired or connected to a smartphone device that includes an installed application associated with the insurance policy of party B. The smart device A 103 and smart device B 104 metadata associated with insurance claim 112 (hereinafter collectively referred to as “insurance claim smart device metadata”) is transmitted through network 114 to server sub-system 102 to be stored by event metadata store mod 304 of FIG. 3.

In this simplified embodiment, metadata from smart device A 103 includes: (i) geolocation data 105 indicating that smart device A was between mile markers 10 and 11 of Route 86 in New York at 3:00 PM EST on Sep. 5, 2019; and (ii) social media data 107 indicating a post was made by party A including information suggesting that they would be travelling along Route 86 in New York during the afternoon of Sep. 5, 2019. Also, in this simplified embodiment, metadata from smart device A 103 includes: (i) heart rate data 106 indicating that party B only experienced one heart rate spike around 3:00 PM EST on Sep. 5, 2019; and (ii) accelerometer data 108 indicating that there was no swerving movement from the arm bearing smart device B 104 and that only one collision occurred suggestive of an object striking the rear of the vehicle of party B after an aggressive deceleration of velocity by the vehicle of party B.

In this simplified embodiment, insurance claim 112 has three primary components, including: (i) insurance claim filed by party A, (ii) insurance claim filed by party B, and (iii) report provided by an insurance company representative. The insurance claims filed by party A and party B comprise information about the insurance claim event, including: (i) date, (ii) time, (iii) location, (iv) accident description, (v) injuries, if any, sustained, (vi) party insurance policy information, and (vii) smart device metadata associated with insurance policy. The report provided by an insurance company representative consists of insurance photos of the vehicles involved in the incident as well as a description of the insurance event from the perspective of an insurance company representative. In this simplified embodiment, the insurance company obtained the insurance claim smart device metadata by an agreement between the respective parties and the insurance company. The agreement stipulates that, in the event of an accident, any smart device metadata associated with a party's insurance policy is to be provided to the insurance company. In exchange, the insurance company agreed to provide each party with a lower monthly auto insurance premium for the party's consent to provide the metadata in the event of an accident.

Alternatively, metadata may be derived from one or more smart devices, including: (a) smartphones, (b) smart speakers, (c) smartwatch, (d) smart rings, (e) smart necklaces, (f) smart glasses, and (g) smart contacts. For example, accelerometer metadata may be transmitted to the cognitive system from a smartphone. As a further alternative embodiment, metadata may be derived from one or more devices for one or more individuals involved in the insurance claim. For example, a person may be involved in a car insurance claim that receives metadata from the persons smartphone and smart watch. As a further alternative embodiment, metadata may be derived from one or more smart medical devices, such as: (a) pacemakers, (b) cybernetic implants, and (c) prosthetic limbs. As a further alternative embodiment, at least one or more types of metadata may be derived from one or more smart devices, including: (a) accelerometer metadata, (b) geolocation metadata, (c) social media account metadata, (d) SMS metadata, (e) phone call metadata, (f) gyroscope metadata, (g) heart rate metadata, (h) eye movement metadata, (i) respiratory metadata, (j) mobile phone application metadata, (k) audio metadata, (l) e-mail metadata, and (m) web-browser metadata. For example, metadata derived from one smartphone may include geolocation, accelerometer, and SMS metadata. Computer systems embedded within vehicles are also available sources of metadata, and may include information such as velocities associated with time stamps, timestamped and intensity of brake engagement, steering angles associated with timestamps, timestamped eye-tracking metadata of the driver, volume level of multimedia output associated with timestamps, etc.

Processing proceeds to operation S265, where metadata analysis mod 306 retrieves stored metadata to be organized by the cognitive system through evaluate metadata sub-mod 308. In this simplified embodiment, the insurance claim smart device metadata is organized by the cognitive system to be utilized by the remaining sub-modules of metadata analysis mod 306. The insurance claim smart device metadata is organized by the type of data being received, and the party and/or parties it is associated with. The metadata derived from smart device A 103 of FIG. 1 is correlated with party A and the auto insurance claim information submitted by party A that is a component of insurance claim 112. The metadata derived from smart device B 104 is correlated with party B and the auto insurance claim information submitted by party B that is a component of insurance claim 112. The insurance claim smart device metadata and insurance claim 112 are processed/structured in a way that is suitable for the cognitive system to analyze the information for potentially fraudulent activity.

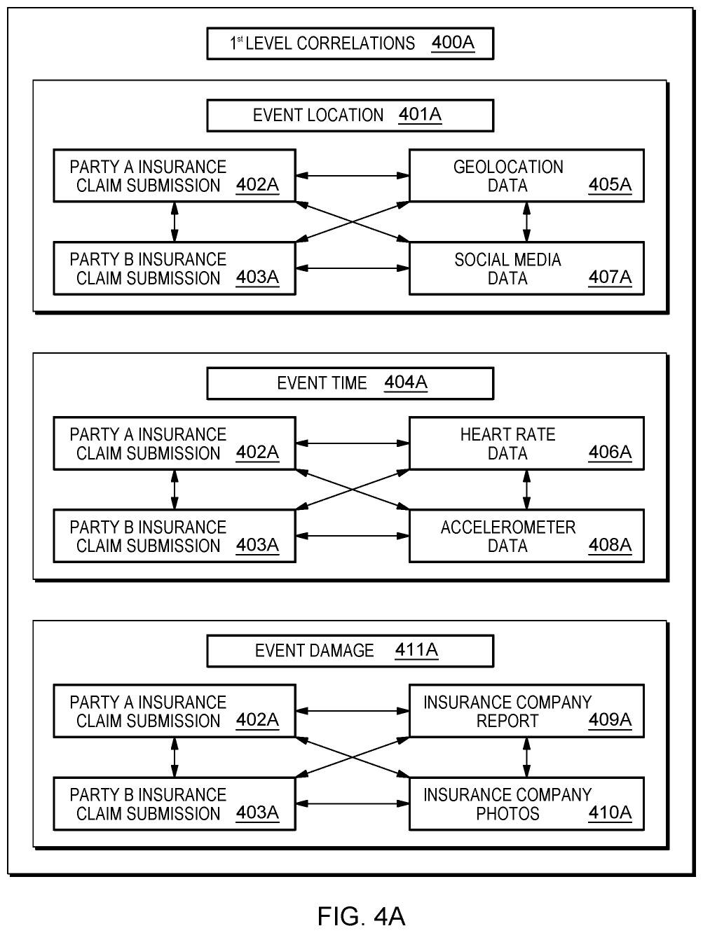

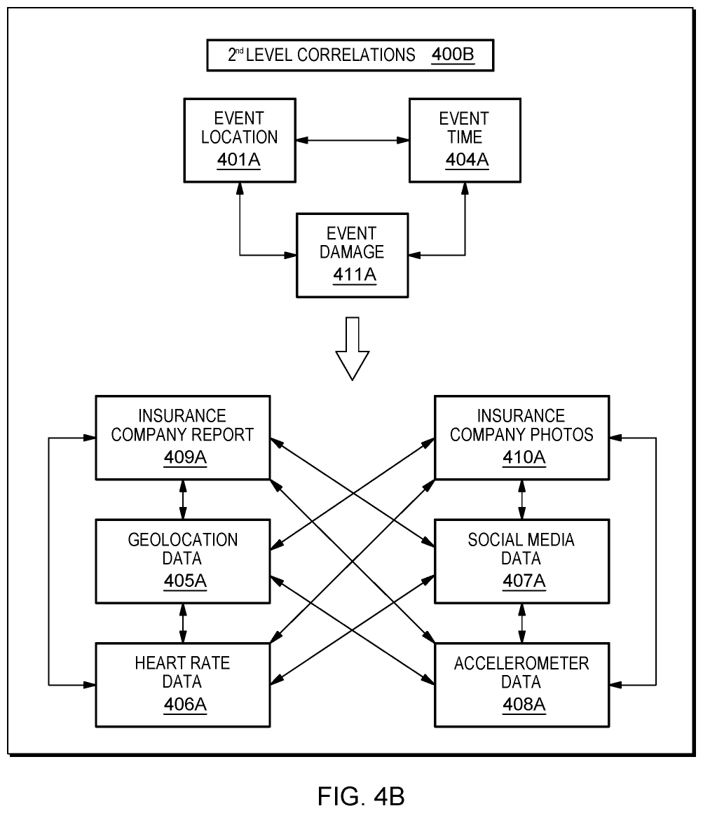

Processing proceeds to operation S270, where generate Pt level correlations sub-mod 310 categorizes data associated with one or more insurance claims to generate cognitive categories to determine further correlations. In this simplified embodiment, the first level correlations 400A of FIG. 4A cognitive categories created, include: (i) event location 401A, (ii) event time 404A, and (iii) event damage 411A.

The event location 401A category consists of the following: (i) party A insurance claim submission 402A, (ii) party B insurance claim submission 403A, (iii) geolocation data 405A, and (iv) social media data 407A. In event location 401A, the party A insurance claim submission 402A and party B insurance claim submission 403A (hereinafter, collectively referred to as “party insurance claim submissions” 402A/403A) are cross-referenced to validate the location of the insurance event based on the information provide in each insurance claim submission regarding the location of the insurance event. In event location 401A, the party insurance claim submissions 402A/403A are cross-referenced with geolocation data 405A to validate the location of the insurance event. In event location 401A, the party insurance claim submissions 402A/403A are cross-referenced with social media data 407A to validate the location of the insurance event. In event location 401A, the geolocation data 405A is cross-referenced with social media data 407A to validate the location of the insurance event. The term cross-referenced, in the context of event location 401A, refers to the comparison of alleged location values of the insurance event, according to four different data sources, to detect inconsistencies and potentially fraudulent activity. For example, if all four data sources of event location 401A category indicate that the accident between party A and party B occurred at 123 Main St. New York, N.Y., then the lack of inconsistencies indicate that the likelihood of fraudulent activity is low, with respect to the location information provided by the four sources. In contrast, if three of the four data sources indicate that the accident between party A and party B occurred at 123 Main St. New York, N.Y., and the fourth data source indicates that the accident occurred at 123 Ocean Ave. Los Angeles, Calif., then the inconsistencies indicate that the fourth source may involve fraudulent activity or information. In this simplified embodiment, each of the data samples for event location 401A all indicate the same event location.

The event time 404A category consists of the following: (i) party A insurance claim submission 402A, (ii) party B insurance claim submission 403A, (iii) heart rate data 406A, and (iv) accelerometer data 408A. In event time 404A, the party insurance claim submissions 402A/403A are cross-referenced to validate the time of the insurance event based on the information provide in each insurance claim submission regarding the time of the insurance event. In event time 404A, the party insurance claim submissions 402A/403A are cross-referenced with heart rate data 406A to validate the time of the insurance event based on a significant change in heart rate to the person wearing smart device B 104 of FIG. 1. In event time 404A of FIG. 4A, the party insurance claim submissions 402A/403A are cross-referenced with accelerometer data 408A to validate the time of the insurance event based on a significant change in accelerometer metadata derived from smart device B 104 of FIG. 1. In event time 404A, the heart rate data 406A of FIG. 4A is cross-referenced with accelerometer data 408A of FIG. 4A to validate the time of the insurance event based on metadata derived from smart device B 104 of FIG. 1. The term cross-referenced, in the context of event time 404A of FIG. 4A, refers to the comparison of alleged time values of the insurance event, according to four different data sources, to detect inconsistencies and potentially fraudulent activity. For example, if all four data sources indicate that the time of the insurance event was at 3:00 PM, then the lack of inconsistencies indicate that the likelihood of fraudulent activity is low, with respect to the time information provided by the four data sources. In contrast, if three of the four data sources indicate that the time of the insurance event was at 3:00 PM, and the fourth data source does not indicate that the insurance event occurred at all because of no significant change in heart beat to the person wearing smart device B 104 of FIG. 1, then the inconsistencies indicate that the fourth source may involve fraudulent activity or information. In this simplified embodiment, within event time 404A, data samples from party A insurance claim submission 402A, heart rate data 406A and accelerometer data 408A support a scenario involving a collision occurring at 3:00 PM EST on Sep. 5, 2019 as a result of one vehicle colliding into the rear of another vehicle as a result of the front vehicle suddenly decelerating without swerving or colliding with an obstacle. Party B insurance claim submission 403A suggests a different timeline of events for a scenario that is inconsistent with the other data samples of event time 404A.

The event damage 411A of FIG. 4A category consists of the following: (i) party A insurance claim submission 402A, (ii) party B insurance claim submission 403A, (iii) insurance company report 409A, and (iv) insurance company photos 410A. In event damage 411A, the party insurance claim submissions 402A/403A are cross-referenced to validate the damage that occurred as a result of the insurance event based on the information provide in each insurance claim submission regarding the resulting damage of the insurance event. In event damage 411A, the party insurance claim submissions 402A/403A are cross-referenced with insurance company report 409A to validate the damage that occurred as a result of the insurance event. In event damage 411A, the party insurance claim submissions 402A/403A are cross-referenced with insurance company photos 410A to validate the damage that occurred as a result of the insurance event. In event damage 411A, the insurance company report 409A is cross-referenced with insurance company photos 410A to validate the damage that occurred as a result of the insurance event. The term cross-referenced, in the context of event damage 411A, refers to the comparison of alleged damage to property that occurred as a result of the insurance event, according to four different data sources, to detect inconsistencies and potentially fraudulent activity. For example, if all four data sources indicate that the insurance event resulted in $2,000 of damage to party A's front bumper and $1,000 of damage to party B's back bumper, then the lack of inconsistencies indicate that the likelihood of fraudulent activity is low, with respect to the insurance event damage information provided by the four data sources. In contrast, if three of the four data sources indicate that the damage to party A's front bumper was $2,000 and the damage to party B's back bumper was $1,000, and a fourth data source provided by party B claims that the insurance event caused $10,000 of damage to party B's back bumper, then the inconsistencies indicate that the fourth data source provided by party B may involve fraudulent activity. In this simplified embodiment

Processing proceeds to operation S275 of FIG. 2, where generate 2^{nd} level correlations sub-mod 312 determines secondary correlations between inputs based on the 1^{st} level correlations generation in operation S270. In this simplified embodiment, second level correlations 400B of FIG. 4B is comprised of an interrelation of the cognitive categories generated in first level correlations 400A of FIG. 4A, including event location 401A of FIG. 4B, event time 404A, and event damage 411A. The interrelated inputs of the cognitive categories generated in first level correlations 400A of FIG. 4A include: (a) insurance company report 409A, (b) insurance company photos 410A, (c) geolocation data 405A, (d) social media data 407A, (e) heart rate data 406A, and (f) accelerometer data 408A. Each of the interrelated inputs is cross-referenced with four other inputs of the interrelated inputs and is not cross-referenced with the interrelated input that were cross-referenced in generate 1^{st} level correlations sub-mod 310 of FIG. 3 to minimize redundancy. The term cross-reference, in the context of second level correlations 400B of FIG. 4B, refers to the interrelation of inputs to compare data types to further detect inconsistencies that may be correlated with fraudulent activity and/or generate inferences between data sources that may be correlated with fraudulent activity.

In this simplified embodiment, the insurance company report 409A of FIG. 4B is cross-referenced with geolocation data 405A and social media data 407A to identify any inconsistencies with the location of the insurance event according to the insurance company report 409A and the location according to geolocation data 405A/social media data 407A. The insurance company report 409A of FIG. 4B is cross-referenced with heart rate data 406A and accelerometer data 408A to identify any inconsistencies with the time of the insurance event according to insurance company report 409A and the time of the insurance event according to heart rate data 406A/accelerometer data 408A. The insurance company photos 410A is cross-referenced with geolocation data 405A and social media data 407A to identify any inconsistencies with the location of the insurance event according to the insurance company photos 410A and the location according to geolocation data 405A/social media data 407A. The insurance company photos 410A is cross-referenced with heart rate data 406A and accelerometer data 408A to identify any inconsistencies with the damage that occurred as a result of the insurance event according to the insurance company photos 410A and the damage that occurred according to heart rate data 406A/accelerometer data 408A. The geolocation data 405A is cross-referenced with accelerometer data 408A to determine if any traffic law violations occurred at the location based on the traffic laws at the location of the insurance event according to geolocation data 405A and the movement of the vehicle at the time of the event according to accelerometer data 408A. The geolocation data 405A is cross-referenced with heart rate data 406A to determine if the location of the insurance event would modify the heart rate of an individual to a point that it would indicate a false-positive of fraudulent activity to the cognitive system. The social media data 407A is cross-referenced with insurance company photos 410A to determine if any photos of one or more vehicles involved in the insurance event were uploaded to social media, and, if so, that the images from social media 407A match insurance company photos 410A. The social media data 407A is cross-referenced with heart rate data 406A to determine if any biometric data was obtained from party A and, if so, the biometric pattern of party A correlates to the biometric pattern, of party B, derived from heart rate data 406A. These are illustrative examples of second level correlations, and other types of second level correlations between similar or different data sources than those discussed above are possible. For example, social media data 407A can be used to extract photographs of property involved in the insurance event prior to the insurance event to verify the extent of damage to the property involved as a result of the insurance event. Machine learning techniques, such as those employed by some embodiments of the present invention, can utilize image processing and computer vision techniques to identify damage that was present prior to the insurance event that is submitted as resulting from the insurance event.

Processing proceeds to operation S280 of FIG. 2, where generate personal risk score sub-mod 314 determines a Personal Risk Score (sometimes hereinafter referred to as “PRS”), for the one or more parties involved in the insurance claim, that indicates the likelihood that a given party is involved in fraudulent activity. In this simplified embodiment, the formula to calculate a party's PRS is the following: PRS=(ΣFA)*50/100, where the variable “FA” is a party's “fraudulent activity.” The summation of fraudulent activity counts multiplied by 50 and divided by 100 determines a party's PRS. An assigned PRS correlates to either a low, medium, or high risk of fraudulent activity. A higher valued PRS indicates a stronger likelihood that a party is involved in some form of fraudulent activity with respect to the submitted insurance claim. A PRS equal to zero (e.g., PRS=0) indicates a low risk that the party is involved in fraudulent activity. A PRS greater than zero and less than one (e.g., 0<PRS<1) indicates a medium risk that the party is involved in fraudulent activity. A PRS greater than or equal to one (e.g., PRS>1) indicates a high risk that the party is involved in fraudulent activity. In this simplified embodiment, the fraudulent activity counts are determined from inconsistencies between interrelated categories and inputs of operation S270 and S275.

In this simplified embodiment, the fraudulent activity (sometimes hereinafter referred to as “FA”) value for party A and party B are determined by the summation of instances, in operation S275 of FIG. 2 and operation S270, that led to inconsistent information between data sources. If a data source originated from a party that was determined to be an inconsistent correlation with other interrelated data sources, then the party responsible for the source would add one count of FA for each inconsistent correlation. The cognitive system does not find any FA instances for party A, according to the data provided by party A through insurance claim 112 of FIG. 1 and metadata derived from smart device A 103. As such, the determined PRS for party A equals zero (i.e., PRS=0), meaning that the cognitive system has determined that party A has a low risk of fraudulent activity.

In this simplified embodiment, the cognitive system determines two instances of FA for party B. The first FA instance is found in the event time 404A of FIG. 4A category of first level correlations 400A. The heart rate data 406A showed that the individual wearing smart device B 104 of FIG. 1 only experienced one significant change in heartbeat during the time period of the insurance event, as opposed to an expected two or more spikes during a scenario where an unexpected obstacle is avoided and results in another collision. The time of a sudden braking and collision insurance event was supported by party A insurance claim submission 402A of FIG. 4A, heart rate data 406A, and accelerometer data 408A. Timing for a swerving maneuver accompanying aggressive braking and a subsequent collision is only supported by party B insurance claim submission 403A. The accelerometer metadata indicates that the car abruptly stopped short, and was moved a short distance indicating impact, at the time of the insurance event as supported by party A insurance claim submission 402A. It is known that spontaneous, dangerous events typically result in suddenly elevated heart rates. The absence of two or more significant changes in heart rate data 406A is inconsistent with the events indicated in party B insurance claim 403A. The inconsistency results in one count of FA to be included in the PRS score of party B and may be indicative of the notion that party B fraudulently anticipated the collision with party A. The second FA instance was determined by the cognitive system in second level correlations 400B of FIG. 4B. The interrelation of insurance company report 409A of FIG. 4B and accelerometer data 408A led to the FA instance. The insurance company report 409A includes an interview by an insurance company representative with party B that claims, “party B swerved to avoid an object that party B perceived to be coming onto the road.” However, the accelerometer data 408A does not indicate any swerving motion at the time of the incident, only an abrupt break prior to the collision. The inconsistent information provided to the insurance company indicates that party B may have fraudulent claimed to have swerved to increase the likelihood of receiving payment for the insurance event. The two instances of FA are applied to the PRS formula described above to result in a PRS equal to one (i.e., PRS=1), meaning that the cognitive system determined that party B has a high risk of fraudulent activity.

Alternatively, the cognitive system and/or an administrator of the cognitive system, may apply a modifier variable to the PRS score based on one or more factors determined to have influence on inputs. For example, the cognitive system may determine that the PRS evaluated is, on average, 20% over the true PRS value and apply a multiplier of 0.80 to the calculated PRS value before categorizing a party's risk level. As a further alternative embodiment, the cognitive system and/or an administrator of the cognitive system, may modify the threshold to categorize a party's risk level. For example, initially a PRS>1 leads to a “high” risk of fraudulent activity and the cognitive system may increase the threshold to only categorize a party as a “high” risk of fraudulent activity when the PRS is greater than 5 (e.g., PRS>5). As a further alternative embodiment, the PRS may have two or more categorizes of fraudulent activity risk. For example, the cognitive system may have five PRS categories of fraudulent activity risk, such as low, low-medium, medium, medium-high, and high. As a further alternative embodiment, individual data sources, such as those woven into an interrelated network in FIG. 4B, may be assigned different weight values for calculating a PRS score. For example, insurance claim forms submitted by the involved parties may be accorded lower relative weight than a police report taken at the scene, or accelerometer data from a wearable device worn by one of the involved parties.

Processing proceeds to operation S285, where process insurance claim mod 316 determines the result of an insurance claim based on the one or more generated Personal Risk Scores. In this simplified embodiment, the cognitive system has a programmed response to a calculated PRS. If a party's PRS is determined to be “low,” then the cognitive system accepts the insurance claim as valid, and proceeds to disperse the agreed upon funds to the party with a low PRS based on the coverage amount stipulated in the party's insurance policy. If a party's PRS is determined to be “medium,” then the cognitive system does not accept the insurance claim as valid, denies the insurance claim, and does not disperse the funds stipulated in the party's insurance policy. If a party's PRS is determined to be “high,” then the cognitive system does not accept the insurance claim as valid, denies the insurance claim, and does not disperse the funds stipulated in the party's insurance policy. In some alternative embodiments, if the cognitive system denies one or more insurance claims (e.g., PRS=medium or PRS=high), then the denied insurance claim is flagged for a representative of the insurance company to further review to confirm that the cognitive system made the appropriate decision. The results of process insurance claim mod 316 are output as described below in operation S290. In this simplified embodiment, because party B's PRS is high, process insurance claim mod 316 automatically generates an insurance claim denial for party B.

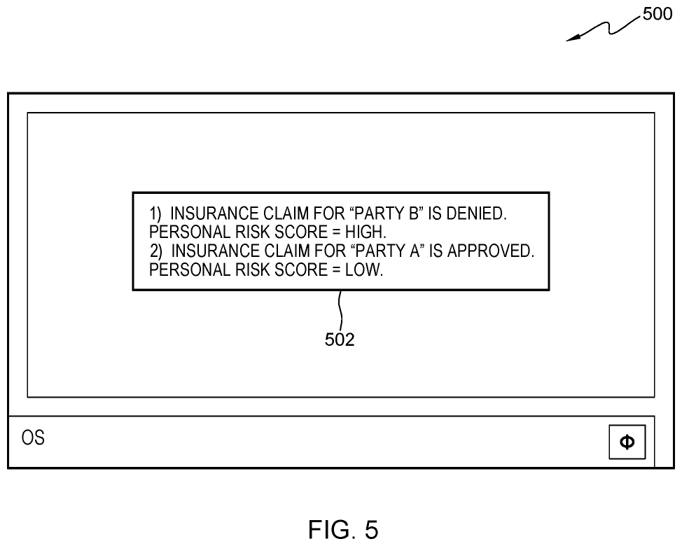

Processing proceeds to operation S290, where result output mod 318 outputs the results and analysis of process insurance claim mod 316 to insurance computer 110. In this simplified embodiment, the first output displays a message 502 of user interface 500 of FIG. 5 that states whether a party to the insurance claim has been approved or denied, as well as the corresponding PRS for the party to the insurance claim. The message 502 is displayed to one or more representatives of the insurance company, for informative purposes, as the claims have already been denied at S285, barring a manually instituted reversal or adjustment by insurance personnel. The second output is a spreadsheet table that explains the steps and results of operation S270, S275, and S280, that were taken by the cognitive system.

### III. Further Comments and/or Embodiments

Some embodiments of the present invention recognize the following facts, potential problems and/or potential areas for improvement with respect to the current state of the art: (i) during the process of insurance, there are situations where the reputation of the individual making the claim is important to determine the claim accuracy; (ii) insurance companies need to have a mechanism to determine the validity of the claim; and (iii) insurance users may want to try to change the real story in order to overcome some regulations of the insurance and obtain insurance payouts, or claimed money (for example, a user may want to switch seats with the driver after an accident in order to apply for the insurance coverage if the driver did not have a valid license to operate the vehicle).

Some embodiments of the present invention may include one, or more, of the following features, characteristics and/or advantages: (i) leverage cognitive technologies to analyze, track and predict risk scenarios that could affect the insurance company during a claim; (ii) a system that analyzes, tracks, and predicts risk scenarios related to insurance claims; (iii) a system that categorizes the metadata related to an insurance claim to create cognitive categories for further correlation; (iv) a system that creates a Personal Risk Score (PRS) based on user patterns of fraudulent activities; (v) evaluate all related metadata to find risky behaviors or characteristics on the claim and use that data to create risk scenarios that may affect the claim; (vi) correlate the information from the user's claim with all available metadata to create risk scenarios; (vii) cognitive system will create categories (date/time, location, injuries, wearables, damages, etc.); (viii) make first level correlations using items within categories to create the “First Level Risk Scenarios”, which has a higher risk score; (ix) make “Second Level Risk Scenarios”, which are based on interrelation items from different categories; and (x) implemented with the user consent and/or insurance company can offer discount rates to users that share data with the insurance company.

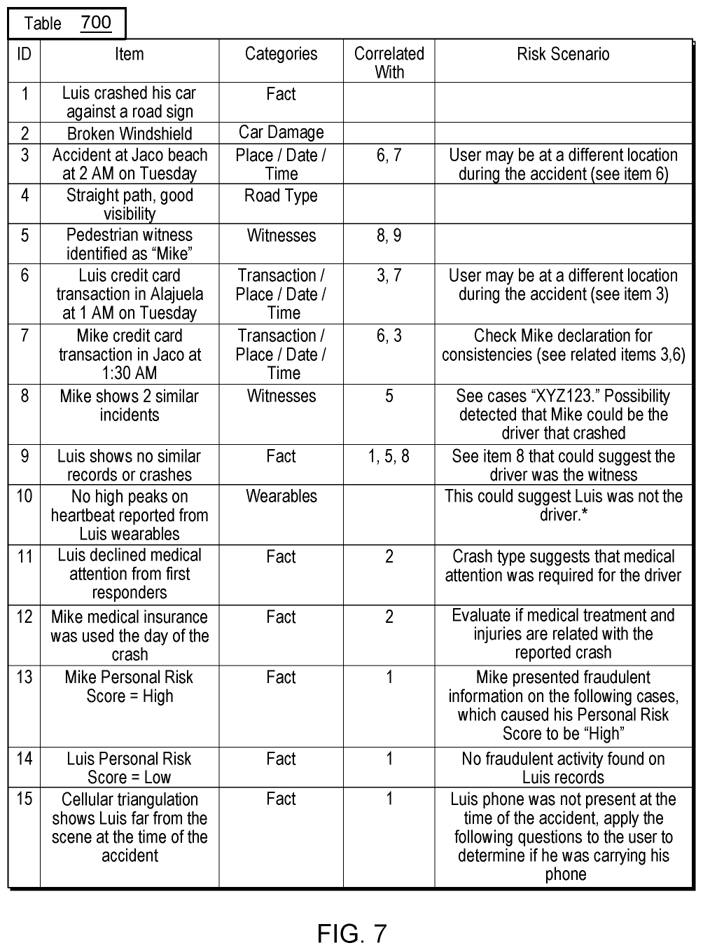

Some embodiments of the present invention may include one, or more, of the following features, characteristics and/or advantages: (i) a calculated Personal Risk Score (PRS) based on previous fraudulent actions (FA) performed by a given person; (ii) a PRS calculated using the following formula: PRS=(ΣFA)*50/100; (iii) a PRS assigned as follows: (a) PRS=0 (Low), (b) PRS<1 (Medium), and (c) PRS>=1 (High) (for example, ID 513 of FIG. 5 assigned “Mike Personal Risk Score=High,” and ID 514 assigned “Luis Personal Risk Score=Low”); (iv) reviewing the insured Personal Risk Score (PRS) and each item from the insurance claim to group it into a category to be correlated with the other item categories to identify inconsistencies between each item in the claim to identify potential risk of fraud (for example, ID 1 of Table 700 of FIG. 7 categorizes the item “Luis crashed his car against a road sign” as a “Fact”); (v) technology to keep record of results; (vi) a Personal Risk Score (PRS) assigned to the implicated party; (vii) an automated cognitive system based on a correlation of claims vs. verifiable facts (for example, ID 5 of Table 700 of FIG. 7 correlates the item “Pedestrian witness identified as Mike” with the items ID 8 and ID 9 of Table 700 of FIG. 7); and (viii) help insurance companies identify potential fraudulent customers through a Personal Risk Score (PRS) based on the user's patterns, which trigger to perform further analysis and correlations to track and predict risk scenarios related to insurance claims (for example, ID 14 of Table 700 of FIG. 7 assigned “Luis Personal Risk Score=Low” based on the risk scenario that determined “No fraudulent activity found on Luis records”.

Some embodiments of the present invention may include one, or more, of the following features, characteristics and/or advantages: (i) help insurance companies identify potential fraudulent customers; (ii) analyze, track, and predict risk scenarios related to insurance claims; (iii) categorize metadata related to an insurance claim to create cognitive categories for further correlation; (iv) create a Personal Risk Score (PRS) based on user patterns of fraudulent activities; (v) a Personal Risk Score (PRS) assigned to all of the parties that were and/or are involved in the insurance accident and/or claim; (vi) in response to receiving metadata associated with an event of a user, evaluating data in the metadata received within a respective category, including: (a) date, (b) time, (c) location, (d) injuries, (e) wearables (e.g., location data, gyroscope data, accelerometer data, heart rate data, etc.), (f) damages, and (g) weather data; (vii) generating a set of first level correlations using items within the respective categories evaluated to create a first level risk scenario for the respective categories; (viii) generating a set of second level correlations using interrelations of the items across different categories evaluated to create a second level risk scenario for the respective categories; (ix) generating a personal risk score using the first level risk scenario, the second level risk scenario and previous fraudulent actions associated with the event of the user, wherein the personal risk score of 0 is identified as low, less than 1 is identified as medium and greater than or equal to 1 is identified as high; and (x) a client identified as making fraudulent claims from different insurance companies.

Some embodiments of the present invention may implement a method which includes some or all of the following steps (not necessarily in the following order): (i) in response to receiving metadata associated with an event of a user, evaluating data in the metadata received within a respective category, including: (a) date, (b) time, (c) location, (d) injuries, (e) wearables (e.g., location data, gyroscope data, accelerometer data, heart rate data, etc.), (f) damages, and (g) weather data; (ii) generating a set of first level correlations using items within the respective categories evaluated to create a first level risk scenario for the respective categories; (iii) generating a set of second level correlations using interrelations of the items across different categories evaluated to create a second level risk scenario for the respective categories; (iv) generating a Personal Risk Score (PRS) using the first level risk scenario, the second level risk scenario and previous fraudulent actions associated with the event of the user; (v) a Personal Risk Score of 0 is identified as low; (vi) a Personal Risk Score less than 1 is identified as medium; and (vi) a Personal Risk Score greater than or equal to 1 is identified as high.

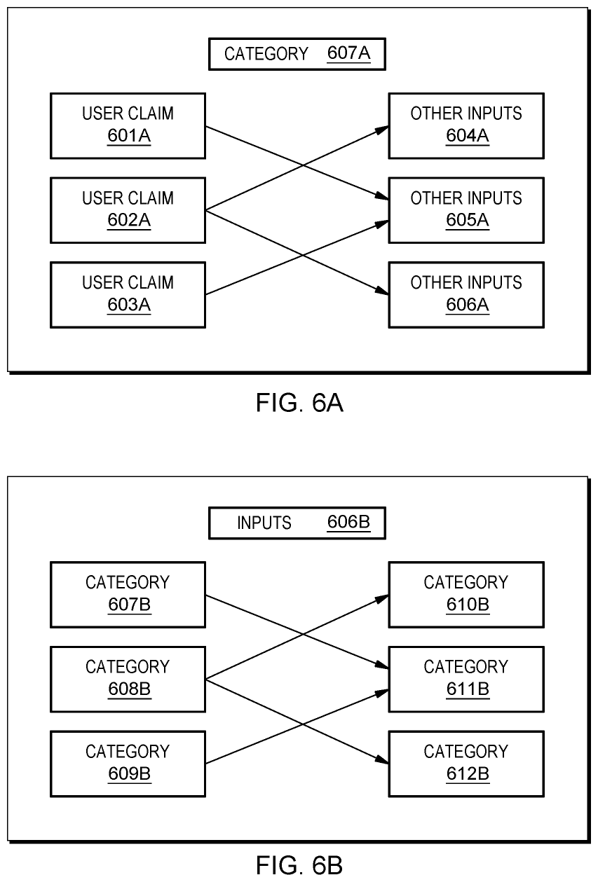

An embodiment of a possible hardware and software environment for software and/or methods according to the present invention will now be described in detail with reference to the Figures. FIG. 6A-6B describes a method of identifying first and second level risk scenarios to quantify the risk of insurance fraud for a given insurance claim. FIG. 6A describes a method to identify first level risk scenarios and includes: (i) category 607A is one category created by a cognitive system; (ii) the cognitive system will create categories, like category 607A, based on available metadata, such as: (a) date/time, (b) location, (c) injuries, (d) wearables (e.g., location data, gyroscope data, accelerometer data, heart rate data, etc.), (e) damages, and (f) weather data, etc.; (iii) category 607A consists of user claim information and available metadata; (iv) user claim information consists of user claim 601A, user claim 602A, and user claim 603A; (v) metadata sources consists of other inputs 604A, other inputs 605A, and other inputs 606A; (vi) the cognitive system generates first level correlations using items within categories; and (vii) generated first level correlations create the first level risk scenarios, which have a higher risk score.

An embodiment of a possible hardware and software environment for software and/or methods according to the present invention will now be described in detail with reference to the Figures. FIG. 6A-6B describes a method of identifying first and second level risk scenarios to quantify the risk of insurance fraud for a given insurance claim. FIG. 6B describes inputs 606B sourced from different categories created from the “First Level Risk Scenarios” and interrelating categories with each other to compare user inputs against hard data to generate “Second Level Risk Scenarios,” and includes: (i) category 607B is sourced from and identical to 607A as described in FIG. 6A; (ii) category 608B is a category created by the cognitive system based on metadata, such as location data; (iii) category 609B is a category created by the cognitive system based on metadata, such as injury data; (iv) category 610B is a category created by the cognitive system based on metadata, such as damages data; (v) category 611B is a category created by the cognitive system based on wearables metadata, such as gyroscope data; (vi) category 612B is a category created by the cognitive system based on wearables metadata, such as accelerometer data; (vii) category 607A and category 609B make correlations with items in category 611B to compare user inputs against hard data to corroborate and/or invalidate user inputs; (viii) category 608B makes correlations with items in category 610B and category 612B to compare user inputs against hard data to corroborate and/or invalidate user inputs; (ix) interrelations of categories may verify and/or invalidate items from other categories; and (x) interrelations determined in the “Second Level Risk Scenario” contribute to generating the overall Personal Risk Score (PRS) assigned to parties involved in the insurance accident/claim.

### IV. Definitions

Present invention: should not be taken as an absolute indication that the subject matter described by the term “present invention” is covered by either the claims as they are filed, or by the claims that may eventually issue after patent prosecution; while the term “present invention” is used to help the reader to get a general feel for which disclosures herein are believed to potentially be new, this understanding, as indicated by use of the term “present invention,” is tentative and provisional and subject to change over the course of patent prosecution as relevant information is developed and as the claims are potentially amended.

Embodiment: see definition of “present invention” above—similar cautions apply to the term “embodiment.”

and/or: inclusive or; for example, A, B “and/or” C means that at least one of A or B or C is true and applicable.

Including/include/includes: unless otherwise explicitly noted, means “including but not necessarily limited to.”

User/subscriber: includes, but is not necessarily limited to, the following: (i) a single individual human; (ii) an artificial intelligence entity with sufficient intelligence to act as a user or subscriber; and/or (iii) a group of related users or subscribers.

Receive/provide/send/input/output/report: unless otherwise explicitly specified, these words should not be taken to imply: (i) any particular degree of directness with respect to the relationship between their objects and subjects; and/or (ii) absence of intermediate components, actions and/or things interposed between their objects and subjects.

Without substantial human intervention: a process that occurs automatically (often by operation of machine logic, such as software) with little or no human input; some examples that involve “no substantial human intervention” include: (i) computer is performing complex processing and a human switches the computer to an alternative power supply due to an outage of grid power so that processing continues uninterrupted; (ii) computer is about to perform resource intensive processing, and human confirms that the resource-intensive processing should indeed be undertaken (in this case, the process of confirmation, considered in isolation, is with substantial human intervention, but the resource intensive processing does not include any substantial human intervention, notwithstanding the simple yes-no style confirmation required to be made by a human); and (iii) using machine logic, a computer has made a weighty decision (for example, a decision to ground all airplanes in anticipation of bad weather), but, before implementing the weighty decision the computer must obtain simple yes-no style confirmation from a human source.

Automatically: without any human intervention.

Module/Sub-Module: any set of hardware, firmware and/or software that operatively works to do some kind of function, without regard to whether the module is: (i) in a single local proximity; (ii) distributed over a wide area; (iii) in a single proximity within a larger piece of software code; (iv) located within a single piece of software code; (v) located in a single storage device, memory or medium; (vi) mechanically connected; (vii) electrically connected; and/or (viii) connected in data communication.

Computer: any device with significant data processing and/or machine readable instruction reading capabilities including, but not limited to: desktop computers, mainframe computers, laptop computers, field-programmable gate array (FPGA) based devices, smart phones, personal digital assistants (PDAs), body-mounted or inserted computers, embedded device style computers, application-specific integrated circuit (ASIC) based devices.

What is claimed is:

1. A computer-implemented method (CIM) comprising:
receiving an insurance event data set, including a plurality of event metadata values;
parsing the event metadata values into a plurality of event data categories;
generate an initial network of correlations between at least some event metadata values within a shared event data category;
generate a secondary network of correlations between at least some event metadata values, where connections are made between event metadata values of different event data categories based, at least in part, on a nature of information corresponding to the event metadata values;
generating a personal risk score (PRS) for one or more involved parties corresponding to an insurance event based, at least in part, on inconsistencies between event metadata values within the initial and secondary networks;
automatically generating an insurance claim conclusion based on one or more PRS scores; and
responsive to automatically generating the insurance claim conclusion, outputting over a computer network to a computer device an electronic message that is modified based on the insurance claim conclusion.

2. The CIM of claim 1, wherein the PRS scores are selected from the group consisting of: (i) low risk, (ii) medium risk, and (iii) high risk.

3. The CIM of claim 2, wherein the automatically generated insurance claim conclusion is a claim denial based, at least in part, on a high risk PRS score.

4. The CIM of claim 1, wherein the outputted electronic message further includes information indicative of how the PRS score was calculated that resulted in the automatically generated insurance claim conclusion.

5. The CIM of claim 1, wherein the plurality of event metadata values includes a heartrate metadata set from a wearable smart device, with the heartrate metadata set including at least one heartrate value associated with a timestamp.

6. The CIM of claim 1, wherein the plurality of event metadata values includes an accelerometer metadata set from a wearable smart device, with the accelerometer metadata set including at least one acceleration value associated with a timestamp.

7. A computer program product (CPP) comprising:
a machine readable storage device; and
computer code stored on the machine readable storage device, with the computer code including instructions for causing a processor(s) set to perform operations including the following:
receiving an insurance event data set, including a plurality of event metadata values;
parsing the event metadata values into a plurality of event data categories,
generate an initial network of correlations between at least some event metadata values within a shared event data category,
generate a secondary network of correlations between at least some event metadata values, where connections are made between event metadata values of different event data categories based, at least in part, on a nature of information corresponding to the event metadata values,
generating a personal risk score (PRS) for one or more involved parties corresponding to an insurance event based, at least in part, on inconsistencies between event metadata values within the initial and secondary networks,
automatically generating an insurance claim conclusion based on one or more PRS scores, and
responsive to automatically generating the insurance claim conclusion, outputting over a computer network to a computer device an electronic message that is modified based on the insurance claim conclusion.

8. The CPP of claim 7, wherein the PRS scores are selected from the group consisting of: (i) low risk, (ii) medium risk, and (iii) high risk.

9. The CPP of claim 8, wherein the automatically generated insurance claim conclusion is a claim denial based, at least in part, on a high risk PRS score.

10. The CPP of claim 7, wherein the outputted electronic message further includes information indicative of how the PRS score was calculated that resulted in the automatically generated insurance claim conclusion.

11. The CPP of claim 7, wherein the plurality of event metadata values includes a heartrate metadata set from a wearable smart device, with the heartrate metadata set including at least one heartrate value associated with a timestamp.

12. The CPP of claim 7, wherein the plurality of event metadata values includes an accelerometer metadata set from a wearable smart device, with the accelerometer metadata set including at least one acceleration value associated with a timestamp.

13. A computer system (CS) comprising:
a processor(s) set;
a machine readable storage device; and
computer code stored on the machine readable storage device, with the computer code including instructions for causing the processor(s) set to perform operations including the following:
receiving an insurance event data set, including a plurality of event metadata values;
parsing the event metadata values into a plurality of event data categories,
generate an initial network of correlations between at least some event metadata values within a shared event data category,
generate a secondary network of correlations between at least some event metadata values, where connections are made between event metadata values of different event data categories based, at least in part, on a nature of information corresponding to the event metadata values,
generating a personal risk score (PRS) for one or more involved parties corresponding to an insurance event based, at least in part, on inconsistencies between event metadata values within the initial and secondary networks,
automatically generating an insurance claim conclusion based on one or more PRS scores, and
responsive to automatically generating the insurance claim conclusion, outputting over a computer network to a computer device an electronic message that is modified based on the insurance claim conclusion.

14. The CS of claim 13, wherein the PRS scores are selected from the group consisting of: (i) low risk, (ii) medium risk, and (iii) high risk.

15. The CS of claim 14, wherein the automatically generated insurance claim conclusion is a claim denial based, at least in part, on a high risk PRS score.

16. The CS of claim 13, wherein the outputted electronic message further includes information indicative of how the PRS score was calculated that resulted in the automatically generated insurance claim conclusion.

17. The CS of claim 13, wherein the plurality of event metadata values includes a heartrate metadata set from a wearable smart device, with the heartrate metadata set including at least one heartrate value associated with a timestamp.

18. The CS of claim 13, wherein the plurality of event metadata values includes an accelerometer metadata set from a wearable smart device, with the accelerometer metadata set including at least one acceleration value associated with a timestamp.
